(19) United States
(12) Patent Application Publication
(10) Pub. No.: US 2022/0031873 A1
(43) Pub. Date: Feb. 3, 2022

(54) Multi-Modal Contrast Agent For Medical Imaging

(71) Applicant: Board of Trustees of Michigan State University, East Lansing, MI (US)

(72) Inventors: Anna V. MOORE, East Lansing, MI (US); Lorenzo F. SEMPERE, DeWitt, MI (US); Neil M. ROBERTSON, Haslett, MI (US)

(73) Assignee: Board of Trustees of Michigan State University, East Lansing, MI (US)

(21) Appl. No.: 17/383,507

(22) Filed: Jul. 23, 2021

Related U.S. Application Data
(60) Provisional application No. 63/058,225, filed on Jul. 29, 2020.

(51) Int. Cl. A61K 51/12; A61B 5/00; A61B 5/055; A61K 51/04; A61K 47/54; A61K 9/50; A61K 49/00

(52) U.S. Cl. CPC A61K 51/1244; A61B 5/0035; A61B 5/0071; A61B 5/0095; A61B 5/055; A61K 49/0093; A61K 51/048; A61K 51/0402; A61K 47/545; A61K 9/5036; A61K 51/0482

(57) ABSTRACT

A nanoparticle is provided. The nanoparticle includes a magnetic core including a magnetic nanocrystal, a fluorophore coupled to the magnetic core, at least one chelating compound coupled to the magnetic core, the at least one chelating compound being a compound that chelates copper-64 (⁶⁴Cu), a compound that chelates technetium-99m (^{99m}Tc), or a combination thereof, and an iodine chelator coupled to the magnetic core. Methods of making the nanoparticle and of using the nanoparticle as a multi-modal contrast agent are also provided.

### CROSS-REFERENCE TO RELATED APPLICATION

This application claims the benefit of U.S. Provisional Application 63/058,225, filed Jul. 29, 2020. The entire disclosure of the above application is incorporated herein by reference.

### FIELD

The present disclosure relates to a contrast agent having a plurality of modalities such that it can be used for a plurality of imaging methods.

### BACKGROUND

This section provides background information related to the present disclosure which is not necessarily prior art.

A contrast agent is a compound used to increase the contrast of naturally occurring or administered cells, structures, or fluids within a body during an imaging procedure. Contrast agents may have various properties depending on the application, location, or imaging technique.

Magnetic resonance imaging (MRI) is a medical imaging technique used to form anatomical images of a subject and/or images of physiological processes in the subject. In MRI, a strong magnetic field is created around an area to be imaged within an MRI scanner. Targeting a selected region, the magnetic field is applied to the subject at a resonant frequency. Atoms of the targeted area are excited and emit a radio frequency signal that is measured by a receiving coil. The signal received by the coil is processed to determine information regarding the scanned area. Contrast agents that interact with the MRI scanner may be given to the subject to make the image clearer.

Magnetic particle imaging (MPI) is an imaging technique that measures a three-dimensional location and concentration of nanoparticle contrast agents to form images. MPI systems use changing magnetic fields to generate a signal from superparamagnetic iron oxide nanoparticles. The changing magnetic fields are used to produce a single magnetic field free region that is moved across a sample. As superparamagnetic iron oxide is not naturally present within a body, a signal is only generated from the administered agent, thus creating an image without background.

Computed tomography (CT), also referred to as computerized axial tomography (CAT), is an imaging technique that uses computer-processed combinations of many X-ray measurements taken from different angles to produce cross-sectional images of specific areas of a scanned object. A contrast agent dye can aid in the visualization of certain structures of interest. CT scans produce data that can be manipulated to demonstrate various bodily structures based on their ability to absorb the X-ray beam.

Positron emission tomography (PET) is a technique that uses radioactive substances to image and measure metabolic processes in the body. PET images are used to detect or measure changes in physiological activities, such as metabolism, blood flow, regional chemical composition, and absorption. The contrast agent used in PET includes radioactive materials that are trapped within tissues of interest. The unstable nucleus of radioligands emits positrons, which combine with neighboring electrons to generate gamma rays. The gamma rays are detected and recorded. Computer reconstruction of the recording produces a three-dimensional image.

Single-photon emission computed tomography (SPECT) is another nuclear medicine tomographic imaging technique that utilizes gamma rays. In SPECT, a radioisotope is attached to a ligand to create a radioligand contrast agent that targets and binds to specific tissues. SPECT is similar to PET in its use of a radioactive material and detection of gamma rays. However, unlike PET, which measures gamma rays produced when positrons contact and combine with electrons, in SPECT, the radioactive material emits gamma radiation that is measured directly.

Fluorescence (FL) imaging is a technique that photographs fluorescent dies and fluorescent proteins, the contrast agents, for visualizing molecular mechanisms and structures. When a certain molecule absorbs light, the energy of the molecule is briefly raised to a higher excited state. The subsequent return to ground state results in an emission of fluorescent light that can be detected and measured.

Photoacoustic (PA) imaging is a technique based on the photoacoustic effect. In PA imaging, after administering a contrast agent, such as a dye, nanostructures comprising gold or carbon, and liposome encapsulations, non-ionizing laser pulses are delivered into tissue where some of the delivered energy is absorbed by the tissue and the contrast agent and converted into heat. The heat leads to transient thermoelastic expansion and wideband ultrasonic emission. Generated ultrasonic waves are detected by ultrasonic transducers, and images are generated from the ultrasonic waves.

Because imaging techniques have differing modes of operation, different contrast agents with specific properties often need to be used to perform different scans in a single subject. Accordingly, a multi-modal contrast agent that can be detected through a plurality of imaging techniques is desired.

### SUMMARY

This section provides a general summary of the disclosure, and is not a comprehensive disclosure of its full scope or all of its features.

The present disclosure relates to a multi-modal contrast agent for medical imaging.

In various aspects, the current technology provides a nanoparticle including a magnetic core having a magnetic nanocrystal; a fluorophore coupled to the magnetic core; at least one chelating compound coupled to the magnetic core, the at least one chelating compound being a compound that chelates copper-64 (⁶⁴Cu), a compound that chelates technetium-99m (^{99m}Tc), or a combination thereof; and an iodine chelator coupled to the magnetic core.

In one aspect, the magnetic nanocrystal includes iron oxide and the magnetic core has a diameter of greater than or equal to about 1 nm to less than or equal to about 50 nm.

In one aspect, the nanoparticle includes a plurality of magnetic cores, wherein each magnetic core of the plurality includes a plurality of magnetic nanocrystals.

In one aspect, the magnetic core is at least partially coated with dextran.

In one aspect, the fluorophore, the at least one chelating compound, and the iodine chelator are indirectly coupled to the magnetic core by way of the dextran.

In one aspect, the fluorophore is selected from the group consisting of a cyanine dye, a coumarin, a rhodamine, a xanthene, a quantum dot, derivatives thereof, and combinations thereof.

In one aspect, the nanoparticle includes the compound that chelates ⁶⁴Cu, the compound that chelates ⁶⁴Cu being S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), or combinations thereof.

In one aspect, the nanoparticle includes the compound that chelates ^{99m}Tc, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA).

In one aspect, the iodine chelator is thyroxine (T4), triiodothyronine (T3), or a combination thereof.

In one aspect, the fluorophore, the compound that chelates ⁶⁴Cu, the compound that chelates ^{99m}Tc, or the combination thereof and the iodine chelator are coupled to the magnetic core by a linker, the linker being aminated epichlorohydrin.

In one aspect, the iodine chelator is further coupled to the core by succinimidyl 6-((beta-maleimidopropionamido)hexanoate) (SMPH) and succinimidyl 3-(2-pyridyldithio)propionate (SPDP).

The current technology also provides a nanoparticle including a single magnetic core having a diameter of greater than or equal to about 20 nm to less than or equal to about 30 nm, the single magnetic core including at least one iron oxide nanocrystal; a coating at least partially covering the single magnetic core, the coating including dextran cross-linked with aminated epichlorohydrin; and an iodine chelator coupled to the aminated epichlorohydrin, the iodine chelator including T4, T3, or a combination thereof, wherein the nanoparticle is configured to provide contrast for MRI, MPI, CT, and X-ray imaging.

In one aspect, the nanoparticle further includes a fluorophore coupled to the single magnetic core, wherein the nanoparticle is further configured to provide contrast for at least one of FL imaging, PA imaging, and ultrasound (US) imaging.

In one aspect, the nanoparticle further includes a compound that chelates ⁶⁴Cu coupled to the single magnetic core, the compound that chelates ⁶⁴Cu being selected from the group consisting of S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), and combinations thereof, wherein the nanoparticle is further configured to provide contrast for PET.

In one aspect, the nanoparticle further includes a compound that chelates ^{99m}Tc coupled to the single magnetic core, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA), wherein the nanoparticle is further configured to provide contrast for SPECT.

In one aspect, the nanoparticle further includes a fluorophore coupled to the single magnetic core; a compound that chelates ⁶⁴Cu coupled to the single magnetic core, the compound that chelates ⁶⁴Cu being selected from the group consisting of S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), and combinations thereof; and a compound that chelates ^{99m}Tc coupled to the single magnetic core, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA), wherein the nanoparticle is further configured to provide contrast for PET, SPECT, and at least one of FL imaging, PA imaging, or US imaging.

The current technology yet further provides a method of making the nanoparticle, the method including reacting iron(II) chloride (FeCl₂) with potassium hydroxide (KOH) in water under the flow of an inert gas to form a reaction mixture, adding hydrogen peroxide (H₂O₂) to the reaction mixture to form a plurality of magnetic cores in the reaction mixture, applying a magnetic field to the reaction mixture and isolating the plurality of magnetic cores, contacting the plurality of magnetic cores with dextran to form a dextran coating at least partially covering each magnetic core of the plurality, cross-linking the dextran with a plurality of epichlorohydrin molecules, aminating the plurality of epichlorohydrin molecules to form the coating including dextran cross-linked with aminated epichlorohydrin on each magnetic core of the plurality, and conjugating a first portion of the aminated epichlorohydrin molecules on at least a portion of the plurality of magnetic cores with the iodine chelator.

In one aspect, the method further includes conjugating a second portion of the aminated epichlorohydrin molecules with a fluorophore, conjugating a third portion of the aminated epichlorohydrin molecules with a compound that chelates ⁶⁴Cu, and conjugating a fourth portion of the aminated epichlorohydrin molecules with a compound that chelates ^{99m}Tc.

In yet various other aspects, the current technology provides a method of imaging at least one of a tissue or a metabolic process in a subject, the method including administering to the subject a safe and effective amount of a composition including a contrast agent, wherein the contrast agent associates with the at least one of the tissue or the metabolic process, the contrast agent including a magnetic core including a magnetic nanocrystal, a fluorophore coupled to the magnetic core, at least one chelating compound coupled to the magnetic core, the at least one chelating compound being a compound that chelates ⁶⁴Cu, a compound that chelates ^{99m}Tc, or a combination thereof, and an iodine chelator coupled to magnetic core; performing at least one of MRI, MPI, FL imaging, PA imaging, US imaging, PET, SPECT, X-ray imaging, or CT; and generating an image of the tissue or the metabolic process with contrast provided by the contrast agent.

In one aspect, the administering is performed intravenously, intraductally, intrathecally, intramuscularly, intratumorally, or orally.

In one aspect, the composition further includes a pharmaceutically acceptable carrier.

In one aspect, the method includes performing at least two of the MRI, the MPI, the FL imaging, the PA imaging, the US imaging, the PET, the SPECT, the X-ray imaging, or the CT and generating at least two images of the tissue or the metabolic process, each of the at least two images having contrast provided by the contrast agent.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustration of a nanoparticle according to various aspects of the current technology.

FIG. 2A is a schematic illustration of a nanoparticle comprising a plurality of magnetic cores in accordance with various aspects of the current technology.

FIG. 2B is a schematic illustration of a nanoparticle including a single magnetic core in accordance with various aspects of the current technology.

FIG. 3 is a reaction scheme for cross-linking a dextran coating with epichlorohydrin and aminating the epichlorohydrin in accordance with various aspects of the current technology.

FIG. 4A is a reaction scheme for modifying aminated magnetic nanoparticles with SPDP in accordance with various aspects of the current technology.

FIG. 4B is a reaction scheme for coupling SMPH to T4 in accordance with various aspects of the current technology.

FIG. 4C is a reaction scheme for coupling the SPDP-modified aminated magnetic nanoparticles of FIG. 4A with the T4-SMPH of FIG. 4B in accordance with various aspects of the current technology.

FIG. 5A is a photograph of a microcentrifuge tube containing nanoparticles prepared in accordance with various aspects of the current technology.

FIG. 5B is an MPI image of the microcentrifuge tube shown in FIG. 5A.

FIG. 5C is an overlay of the images from FIG. 5A and FIG. 5B.

FIG. 6A is a photograph of a microcentrifuge tube containing nanoparticles prepared in accordance with various aspects of the current technology.

FIG. 6B is an MPI image of the microcentrifuge tube shown in FIG. 6A.

FIG. 7A is a first MRI image obtained from a first exposure (7.01 ms) of various samples, wherein Samples A, B, and C include nanoparticles prepared in accordance with various aspects of the current technology, Sample D contains nanoparticles that are not prepared in accordance with the current technology, and Sample E is water.

FIG. 7B is a second MRI image obtained from a second exposure (11.03 ms) of the various samples described with reference to FIG. 7A.

FIG. 8 is a fluorescence image of a microcentrifuge tube containing nanoparticles prepared in accordance with various aspects of the current technology.

FIG. 9 is a photoacoustic image generated by multispectral optoacoustic tomography (MSOT) of a sample including nanoparticles prepared in accordance with various aspects of the current technology and of a water sample.

FIG. 10 is an ultrasound image generated by MSOT of a sample including nanoparticles prepared in accordance with various aspects of the current technology and of a water sample.

FIG. 11A shows two-dimensional CT images of a microcentrifuge tube containing nanoparticles prepared in accordance with various aspects of the current technology.

FIG. 11B shows a three-dimensional CT image of the microcentrifuge tube described with reference to FIG. 11A.

FIG. 12A shows overlaid MPI and CT images of a mouse that had an intraductal administration of nanoparticles prepared in accordance with various aspects of the current technology.

FIG. 12B shows a first two-dimensional MPI/CT overlaid image of the mouse of FIG. 12A.

FIG. 12C shows a second two-dimensional MPI/CT overlaid image of the mouse of FIG. 12A.

FIG. 12D shows a third two-dimensional MPI/CT overlaid image of the mouse of FIG. 12A.

FIG. 13A is a fluorescence image of a first mouse that had an intraductal administration of nanoparticles prepared in accordance with various aspects of the current technology. The mouse is the same mouse as described in FIGS. 12A-12D.

FIG. 13B is a fluorescence image of a second mouse that had an intraductal administration of nanoparticles prepared in accordance with various aspects of the current technology.

FIG. 14A shows two-dimensional CT images of a mouse that had an intraductal administration of nanoparticles prepared in accordance with various aspects of the current technology. The mouse is the same mouse as described in FIGS. 12A-12D and 13A.

FIG. 14B shows a first three-dimensional CT image of the mouse of FIG. 14A.

FIG. 14C shows a second three-dimensional CT image of the mouse of FIG. 14A.

FIG. 15A shows a first fluorescence image of a mouse that had an intravenous administration of nanoparticles made in accordance with various aspects of the current technology as a contrast agent.

FIG. 15B shows a second fluorescence image of the mouse shown in FIG. 15A.

FIG. 15C is a bioluminescence image of the mouse of FIG. 15A, where the bioluminescence indicates a tumor.

FIG. 16A is an image after omni particle intraductal injection of a mouse specimen showing a localization of a PET-only signal within the fat pad, specifically within the ductal tree network.

FIG. 16B shows a PET and MRI overlaid image of the mouse specimen of FIG. 16A.

FIG. 16C shows an MRI-only image of the mouse specimen of FIG. 16A.

Corresponding reference numerals indicate corresponding parts throughout the several views of the drawings.

### DETAILED DESCRIPTION

Example embodiments are provided so that this disclosure will be thorough, and will fully convey the scope to those who are skilled in the art. Numerous specific details are set forth such as examples of specific compositions, components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps, processes, and operations described herein are not to be construed as necessarily requiring their performance in the particular order discussed or illustrated, unless specifically identified as an order of performance. It is also to be understood that additional or alternative steps may be employed, unless otherwise indicated.

When a component, element, or layer is referred to as being “on,” “connected to,” or “coupled to” another element, it may be directly on, connected or coupled to the other element, or intervening elements may be present. In contrast, when an element is referred to as being “directly on,” “directly connected to,” or “directly coupled to” another element or layer, there may be no intervening elements present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges.

Example embodiments will now be described more fully with reference to the accompanying drawings.

Because imaging techniques have differing modes of operation, different contrast agents with specific properties often need to be used to perform different imaging methods in a single subject. Typically, when a subject is in need of imaging, a contrast agent is administered to the subject, wherein the contrast agent is configured to provide contrast for a specific imaging method. The specific imaging method is then performed. If the subject is in need of a second different type of imaging, a second contrast agent is administered to the subject, wherein the second contrast agent is configured to provide contrast for a second specific imaging method that is different form the first specific imaging method. The second imaging method is then performed. This pattern is repeated for each different type of imaging that is performed on the subject. When more than one type of imaging is desired, separate administering and imaging steps require long periods of time and may result in added stress and anxiety to the subject. Accordingly, the current technology provides a multi-modal contrast agent that can be detected through a plurality of imaging techniques after a single administration. The multi-modal contrast agent has a magnetic nanoparticle core and detectable agents coupled to the magnetic nanoparticle core.

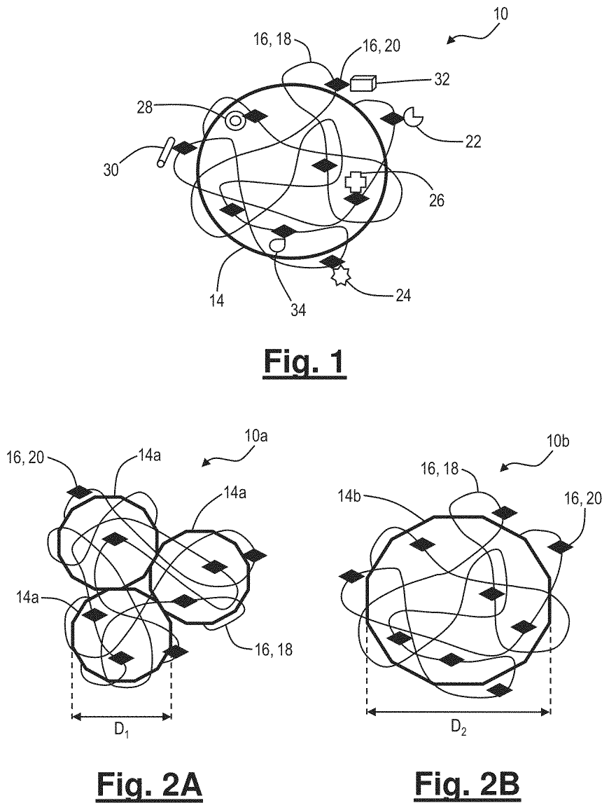

With reference to FIG. 1, the current technology provides a nanoparticle 10 that is configured to provide contrast for a plurality of imaging methods. The nanoparticle 10 comprises at least one magnetic core 14, wherein the at least one magnetic core comprises at least one magnetic nanocrystal, such a plurality of magnetic nanocrystals. The at least one magnetic nanocrystal is diamagnetic, paramagnetic, superparamagnetic, or ferromagnetic and includes iron oxide, which may be iron(II) oxide (FeO), iron(III) oxide (Fe₃O₄), or a combination or network thereof. As such, the at least one magnetic core 14 is diamagnetic, paramagnetic, superparamagnetic, or ferromagnetic and comprises the iron oxide. As a non-limiting example, the at least one magnetic core 14 can be at least one superparamagnetic iron oxide nanoparticle. The at least one magnetic core 14 has a diameter of greater than or equal to about 1 nm to less than or equal to about 50 nm, as discussed in more detail below. The at least one magnetic core 14 is a detectable agent in that it provides contrast for MRI and MPI.

The nanoparticle 10 further comprises a coating 16 covering the at least one magnetic core 14. The coating 16 at least partially covers the at least one magnetic core 14 and may substantially cover the entirety of the at least one magnetic core 14. The term “substantially cover the entirety of the at least one magnetic core 14” means that at least 80% of each magnetic core 14 is covered by the coating 16. The coating 16 comprises a polymer 18 having at least one functional group 20. The at least one functional group 20 is selected from the group consisting of an amine (e.g., amino), a thiol (also referred to as a sulfanyl group or a sulfhydryl group), a hydroxyl, a carboxyl, a phosphate, an aldehyde, an azide, a vinyl (e.g., vinyl sulfone), an alkyne/dibenzocyclooctyne (DBCO), a lysine, a maleimide, biotin, and combinations thereof, as non-limiting examples. The at least one functional group 20 is a part of at least one of the backbone of the polymer 18, i.e., within the polymer 18 itself, or a cross-linking agent that cross-links the polymer 18. The at least one functional group 20 is configured to couple, i.e., conjugate, detectable agents to the at least one magnetic core 14. As used herein, “detectable agents” are compounds that provide contrast for imaging methods. The at least one functional group 20 can be used for “click chemistry” to conjugate at least one detectable agent to the at least one magnetic core 14. The polymer 18 can be dextran (having an average molecule weight of greater than or equal to about 1000 g/mol to less than or equal to about 100,000 g/mol; such as dextran T1, T1.5, G3.5, T5, T6, T10, T20, T25, T40, T60, T70, or T110), polyethylene glycol (PEG), poly ethyl methacrylate, poly methyl methacrylate, functionalized derivatives thereof, or combinations thereof, as non-limiting examples. When present, the cross-linker can be epichlorohydrin, N,N′-methylenebis(acrylamide), functionalized derivatives thereof, or combinations thereof, as non-limiting examples. In some aspects, the polymer 18 comprises dextran that is modified to have at least one functional group 20 or unmodified dextran that is cross-liked with a cross-linking agent comprising the at least one functional group 20, such as aminated, thioloated, or carboxylated epichlorohydrin.

As discussed above, the nanoparticle 10 comprises at least one magnetic core 14. The at least one magnetic core 14 is substantially spherical or semi-spherical. As used herein, “substantially spherical” means that the at least one magnetic core 14 may not be a perfect sphere and may include surface, i.e., topological, irregularities, such as crests, dips, flat portions, and the like.

In various aspects, and with reference to FIG. 2A, the nanoparticle 10 is a nanoparticle 10a comprising at least two, i.e., a plurality of, magnetic cores 14a. For example, the nanoparticle 10a can include from 2 to about 20 or more magnetic cores 14a. Each magnetic core 14a of the plurality has a diameter D₁ of greater than or equal to about 1 nm to less than or equal to about 15 nm. As such, the diameter of the nanoparticle 10a is dependent on the number of magnetic cores 14a present and on the thickness of the coating 16. The plurality of magnetic cores 14a are collectively at least partially covered or coated with the coating 16 as described above.

In various other aspects, and with reference to FIG. 2B, the nanoparticle 10 is a nanoparticle 10b comprising only a single magnetic core 14b. The single magnetic core 14b has a diameter D₂ of greater than or equal to about 15 nm to less than or equal to about 50 nm or greater than or equal to about 20 nm to less than or equal to about 30 nm. The single magnetic core 14b is at least partially covered or coated with the coating 16 as described above.

Referring back to FIG. 1, the nanoparticle 10 comprises at least one additional detectable agent coupled to the at least one magnetic core 14, the at least one magnetic core 14 itself being a detectable agent, as discussed above. The at least one additional detectable agent can be an iodine chelator 22, a fluorophore 24, a chelating compound that chelates ⁶⁴Cu 26, a chelating compound that chelates ^{99m}Tc 28, or combinations thereof. Although FIG. 1 shows each of the iodine chelator 22, the fluorophore 24, the chelating compound that chelates ⁶⁴Cu 26, and the chelating compound that chelates ^{99m}Tc 28, it is understood that the nanoparticle 10 includes at least one of these detectable agents. In some aspects, the nanoparticle 10 comprises the iodine chelator 22 and optionally another detectable agent selected from the group consisting of the fluorophore 24, the chelating compound that chelates ⁶⁴Cu 26, the chelating compound that chelates ^{99m}Tc 28, and combinations thereof. In other aspects, the nanoparticle 10 comprises at least two additional detectable agents selected from the group consisting of the iodine chelator 22, the fluorophore 24, the chelating compound that chelates ⁶⁴Cu 26, and the chelating compound that chelates ^{99m}Tc 28, and combinations thereof. The at least one detectable agent is coupled directly to the at least one functional group 20 provided by the cross-linking agent or the polymer 18 or indirectly to the cross-linking agent or the polymer 18 by way of a linker.

In some aspects, the nanoparticle 10 comprises the iodine chelator 22. The iodine chelator 22 is a compound that chelates iodine or otherwise binds iodine such that the iodine is detectable, i.e., provides contrast, for CT. The iodine chelator 22 binds to the at least one functional group 20 directly or indirectly by way of at least one linker. As non-limiting examples, the iodine chelator 22 can be T4, T3, or a combination thereof. Conventional iodine tracers can also be used as the iodine chelator 22. In certain aspects, the iodine chelator 22 is coupled to the at least one functional group 20 on the cross-linking agent by the linkers SMPH and SPDP. When present, the nanoparticle 10 provides contrast for MRI and MPI by way of the at least one magnetic core 14 and for X-ray imaging and CT by way of the iodine bound to the iodine chelator 22. In some aspects, the iodine chelator 22 is present on the nanoparticle 10 in greater than or equal to about 3 to less than or equal to about 20 or more copies or molecules.

In some aspects, the nanoparticle 10 comprises the fluorophore 24. The fluorophore 24 is a compound that luminesces when stimulated by certain wavelengths of light and provides contrast for at least one of FL imaging, PA imaging, or US imaging. As non-limiting examples, the fluorophore 24 can be a cyanine dye, a coumarin, a rhodamine, a xanthene (e.g., fluorescein and rhodamine), a quantum dot, derivatives thereof, and combinations thereof. Non-limiting examples of cyanine dyes include Cy3, Cy3.5, Cy5, Cy5.5, Cy7, Cy7.5, and combinations thereof. Derivatives of the fluorophore 24 include a base fluorophore compound that is modified to improve at least one of performance, for example, by minimizing photobleaching and pH-dependent fluorescent intensity, or coupling to the at least one functional group 20. The modified base fluorophore can be sulfated, isothiocyanated, modified with an N-hydroxysuccinimide (NHS) ester, modified with a maleimide, modified with biorthogonal conjugates, such as azide, azido, alkyne, tetrazine, or alkene, or otherwise modified to improve performance or coupling. Non-limiting examples of commercially available fluorophores include Alexa Fuor® fluorescent dyes commercialized by Thermo Fisher Scientific, Dylight™ fluorescent dyes, and Opal™ fluorescent dyes commercialized by PerkinElmer. When present, the nanoparticle 10 provides contrast for MRI and MPI by way of the at least one magnetic core 14 and for at least one of FL imaging, PA imaging, or US imaging by way of the fluorophore 24. In some aspects, the fluorophore 24 is present on the nanoparticle 10 in greater than or equal to about 1 to less than or equal to about 15 or more copies or molecules.

In some aspects, the nanoparticle 10 comprises the chelating compound that chelates ⁶⁴Cu 26. The chelating compound that chelates ⁶⁴Cu 26 is a compound that chelates ⁶⁴Cu or that otherwise binds ⁶⁴Cu such that the ⁶⁴Cu is detectable, i.e., provides contrast, for PET. The chelating compound that chelates ⁶⁴Cu 26 binds to the at least one functional group 20 directly or indirectly by way of at least one linker. As non-limiting examples, the chelating compound that chelates ⁶⁴Cu 26 can be S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), or combinations thereof. When present, the nanoparticle 10 provides contrast for MRI and MPI by way of the at least one magnetic core 14 and for PET by way of the compound that chelates ⁶⁴Cu 26.

In some aspects, the nanoparticle 10 comprises the chelating compound that chelates ^{99m}Tc 28. The chelating compound that chelates ^{99m}Tc 28 is a compound that chelates ^{99m}Tc or that otherwise binds ^{99m}Tc such that the ^{99m}Tc is detectable, i.e., provides contrast, for SPECT. The chelating compound that chelates ^{99m}Tc 28 binds to the at least one functional group 20 directly or indirectly by way of at least one linker. As a non-limiting example, the chelating compound that chelates ^{99m}Tc 28 can be [(R)-2-amino-3-(4-isothiocyanatophenyl)propyl]-trans-(S,S)-cyclohexane-1,2-diamine-pentaacetic acid (SCN-Bn-CHx-DTPA). When present, the nanoparticle 10 provides contrast for MRI, MPI, and X-ray imaging by way of the at least one magnetic core 14 and for SPECT by way of the chelating compound that chelates ^{99m}Tc 28.

The nanoparticle 10 is configured as a multi-modal contrast agent because it includes the at least one magnetic core 14 (which provides contrast for MRI and MPI) and at least one of the iodine chelator 22 (which provides contrast for X-ray imaging and CT), the fluorophore 24 (which provides contrast for at least one of FL imaging, PA imaging, or US imaging), the chelating compound that chelates ⁶⁴Cu 26 (which provides contrast for PET), or the chelating compound that chelates ^{99m}Tc 28 (which provides contrast for SPECT). When the nanoparticle 10 comprises each of the iodine chelator 22, the fluorophore 24, the chelating compound that chelates ⁶⁴Cu 26, and the chelating compound that chelates ^{99m}Tc 28, a subject receiving a single administration of a composition comprising the nanoparticle 10 can sequentially undergo MRI, MPI, X-ray imaging, CT, at least one of FL imaging, PA imaging, or US imaging, PET, and SPECT, either during a single day or on different days within about 2 weeks without receiving a second administration of the composition or of another contrast agent. It is understood that the nanoparticle 10 can be used to provide contrast for MRI and MPI, along with at least one of X-ray imaging, CT, FL imaging, PA imaging, US imaging, PET, and SPECT, depending on the at least one detectable agent that is coupled to the at least one magnetic core 14 of the nanoparticle 10.

In some aspects, the nanoparticle also optionally includes an adjunct agent coupled to the at least one magnetic core 14. As non-limiting examples, the adjunct agent can be an oligonucleotide 30, a protein or peptide 32, an active pharmaceutical ingredient 34, or combinations thereof.

The current technology also provides a composition comprising the nanoparticle 10. The composition further comprises a pharmaceutically acceptable carrier suitable for intravenous, intraductal, intrathecal, intramuscular, intratumorally, or oral administration. Thus, the composition can be a liquid, a gel, or a solid, such as a tablet or capsule. The pharmaceutically acceptable carrier can include water, a saline solution, a preservative, a binder, or combinations thereof, as non-limiting examples.

The current technology also provides a method of making the nanoparticle 10. The method comprises forming the magnetic cores 14a described with reference to FIG. 2A or the single magnetic core 14b described with reference to FIG. 2B.

The method of making the nanoparticle 10a comprising the plurality of magnetic cores 14a having a polymeric coating comprises dissolving a polymer in water to a polymer concentration of greater than or equal to about 15% to less than or equal to about 40%, optionally in an ice bath, to form a polymer solution. Next, the method comprises flowing an inert gas through the polymer solution, such as nitrogen, neon, or argon, as non-limiting examples, and adding iron(III) chloride hexahydrate (FeCl₃.6H₂O) to a final concentration of greater than or equal to about 0.5% to less than or equal to about 5%. Next, the method comprises adding iron(II) chloride hexahydrate (FeCl₂.4H₂O) to a final concentration of greater than or equal to about 0.25% to less than or equal to about 2.5%. Next, the method comprises adding NH₄OH to a final concentration of greater than or equal to about 20% to less than or equal to about 40% and increasing the temperature to greater than or equal to about 70° C. to less than or equal to about 90° C. for greater than or equal to about 5 minutes to less than or equal to about 2 hours or longer. The method then comprises concentrating the solution to a final volume that is about 60% of the previous volume and adding NaOH to a final concentration of greater than or equal to about 1 M to less than or equal to about 3 M and greater than or equal to about 10% to less than or equal to about 25% of concentrated (e.g., 90-99%) cross-linker and stirring for greater than or equal to about 1 hour to less than or equal to about 24 hours. The cross-linker can be aminated as described below.

In some aspects, the method comprises mixing about 18 g of Dextran-T10 with about 60 mL of double-distilled water and stirring in a round bottom flask in an ice bath until dissolved completely and a clear dextran suspension is formed. Then, the method comprises adding 1.3 g of FeCl₃.6H₂O into the clear dextran suspension while flushing argon gas into the reaction mixture. Next, the method comprises dissolving 0.8 g of FeCl₂.4H₂O in 5 mL of distilled water, which was previously flushed with argon gas in order to avoid oxidation, and adding this solution into the clear dextran suspension to form a reaction mixture. The method then comprises adding about 30 mL of concentrated cold NH₄OH (approximately 28%) to the reaction mixture and increasing the temperature to from about 75° C. to about 85° C. for about one hour. Next, the method comprises cooling to room temperature and concentrating to about 40 mL using centrifugal units (MWCO 100 kDa).

The method of making the nanoparticle 10b comprising the single magnetic core 14b comprises combining a magnetic compound source, such as iron(II) chloride hexahydrate as a non-limiting example, with water to form a solution comprising greater than or equal to about 0.5% to less than or equal to about 10% of the iron magnetic compound source. The resulting solution is stirred at about atmospheric temperature and pressure for greater than or equal to about 0.5 minutes to less than or equal to about 20 minutes to allow for oxygen equilibrium. The method then comprises introducing an inert gas, such as nitrogen, neon, or argon, as non-limiting examples, to the oxygen-equilibrated solution for greater than or equal to about 1 minute to less than or equal to about 60 minutes. A base, such as NaOH or KOH, is then added to a final concentration of greater than or equal to about 0.5 M to less than or equal to about 1 M, and the reaction is maintained for greater than or equal to about 1 minute to less than or equal to about 30 minutes. Thus, the method comprises forming the magnetic nanoparticles by reacting a magnetic compound source with a base in water under the flow of an inert gas to form a reaction mixture.

Next, the method comprises introducing a first addition of H₂O₂ to the reaction mixture to a final concentration of greater than or equal to about 0.005% to less than or equal to about 0.1% and allowing the solution to react for greater than or equal to about 0.5 minutes to less than or equal to about 20 minutes. A second addition of H₂O₂ is then introduced to the reaction mixture to the same final concentration, and the reaction mixture is again allowed to react for greater than or equal to about 0.5 minutes to less than or equal to about 20 minutes to complete the formation of the magnetic nanoparticles, which can be separated from the reaction mixture with a magnet.

The method also comprises disposing the coating 16 on the at least one magnetic core 14. Here, the method comprises suspending the at least one magnetic core 14 in water, adding a polymer to the suspension to a final concentration of greater than or equal to about 2% to less than or equal to about 10%, and stirring the suspension for greater than or equal to about 1 minute to less than or equal to about 60 minutes. The suspension is then brought to a temperature of greater than or equal to about 50° C. to less than or equal to about 100° C. under the flow of an inert gas and maintained for greater than or equal to about 1 hour to less than or equal to about 24 hours. Then, the method comprises bringing the suspension to ambient or room temperature, magnetically separating the at least one magnetic core 14 to concentrate the suspension, and filtering the suspension to remove excess polymer and to form the polymer 18 of the coating 16.

When necessary, the method comprises cross-linking the polymer 18 with the cross-linking agent comprising the at least one functional group 20. Here, the method comprises adding a base, e.g., NaOH or KOH, to the suspension comprising the at least one magnetic core 14 at least partially coated with the polymer 18 to a final concentration of greater than or equal to about 0.1 M to less than or equal to about 1 M and mixing for greater than or equal to about 1 minute to less than or equal to about 1 hour. Next, the method comprises adding a cross-linking agent to the suspension and mixing for greater than or equal to about 1 hour to less than or equal to about 24 hours to form the coating 16 comprising the polymer 18 and the cross-linking agent on a least a portion of the at least one magnetic core 14.

The polymer 18 or the cross-linking agent, when present, can be modified to include the at least one functional group 20. The following description is based on aminating the cross-linking agent to result in a plurality of aminated cross-linking molecules. However, other modifications can be made using methods known in the art.

Aminating is performed by adding concentrated NH₄OH, e.g., from about 20% to about 40% NH₄OH, to the suspension to a final NH₄OH concentration of from about 1% to about 15% and stirring for greater than or equal to about 1 hour to less than or equal to about 24 hours. The suspension is then membrane purified, i.e., dialyzed, to remove small molecules, and the at least one magnetic core 14 is concentrated by magnetic separation.

In certain aspects, the polymer 18 comprises dextran, and the cross-linking agent comprises aminated epichlorohydrin.

The method further comprises conjugating a first portion of the aminated cross-linking agent molecules, or other molecules of the polymer 18 that include the at least one functional group 20, with a detectable agent.

In some aspects, the method comprises conjugating a portion of the aminated epichlorohydrin molecules with the iodine chelator 22, such as by contacting the aminated cross-linking agent with SPDP to form cross-linking agent molecules comprising SPDP, contacting an iodine chelator with SMPH to form an iodine chelator comprising SMPH, and contacting the cross-linking agent molecules comprising SPDP with the iodine chelator comprising SMPH to complete the conjugation.

In some aspects, the method comprises conjugating a portion of the aminated epichlorohydrin molecules with the fluorophore 24, such as by contacting the aminated cross-linking agent with a fluorophore having an NHS moiety.

In some aspects, the method comprises conjugating a portion of the aminated epichlorohydrin molecules with the chelating compound that chelates ⁶⁴Cu 26, such as by contacting the aminated cross-linking agent with a chelating compound that chelates ⁶⁴Cu having an NHS moiety.

In some aspects, the method comprises conjugating a portion of the aminated epichlorohydrin molecules with the chelating compound that chelates ^{99m}Tc 28, such as by contacting the aminated cross-linking agent with a chelating compound that chelates ^{99m}Tc having an NHS moiety.

The current technology yet further provides a method of imaging at least one of a tissue or a metabolic process in a subject in need thereof. The method comprises administering to the subject a safe and effective amount of a composition comprising the nanoparticle 10 (or plurality of nanoparticles 10) discussed above, wherein the nanoparticle 10 is a multi-modal contrast agent. As used herein, the term “effective amount” means an amount of a compound which, when administered to a subject, is sufficient to provide contrast for each imaging method being performed on the subject. The “effective amount” will vary depending on, for example, the composition form (e.g., the carriers present), the nanoparticle form (i.e., the detectable agents present), the dosage form, the condition requiring imaging, the anatomical region requiring imaging, and the age and weight of the patient to be subjected to the imaging. In various aspects, the effective amount of the composition provides a dose of greater than or equal to about 1 mg/kg to less than or equal to about 100 mg/kg. As used herein, the “subject” is a human or non-human mammal, a bird, a reptile, or an amphibian. The administering can be performed intravenously, intraductally, intrathecally, intramuscularly, intratumorally, or orally.

Depending on the detectable agents present on the nanoparticle 10, the method further comprises performing at least one or at least two of MRI, MPI, FL imaging, PA imaging, US imaging, PET, SPECT, X-ray imaging, or CT on the subject and generating at least one or at least two images of the tissue or the metabolic process with contrast provided by the nanoparticle 10.

Embodiments of the present technology are further illustrated through the following non-limiting example.

### Example

A multi-modal magnetic nanoparticle contrast agent is prepared as follows.

### Coated Magnetic Nanoparticle Synthesis

Iron oxide nanoparticles are synthesized with a newly adapted co-precipitation technique. Briefly, 2.0 g of iron(II) chloride hexahydrate are dissolved in 100 mL of deionized (DI) water with stirring at atmospheric temperature and pressure for 5 minutes to allow for oxygen equilibrium. Afterwards, nitrogen gas flow is started and allowed to flow for 15 minutes before initiation of the reaction. 20 mL of a 5 M KOH solution is then added to the solution and mixed for 10 minutes under the nitrogen gas flow. To this solution, 250 μL of a 5% H₂O₂ in DI water is added twice, with 5 minutes between the additions. 5 minutes after the second addition of 5% H₂O₂ in DI, the solution is removed from nitrogen flow and capped to undergo magnetic separation. A potent magnetic field is applied for about 20 minutes to fully pull the nanoparticles out of solution and the supernatant is removed. The nanoparticles are resuspended in 70 mL of DI water and allowed to stir for 5 minutes.

After the stirring, 4 g of dextran T-10 is added to the solution and it is again allowed to stir for 15 minutes. This mixture of nanoparticles and dextran is then brought to 85° C. under nitrogen flow and kept there for 8 hours. After the 8 hour incubation, the particles are removed from the heat and brought to room temperature. They are again magnetically separated to concentrate the solution, followed by purification with Millipore® filter devices to remove excess dextran. 7 mL of nanoparticle solution is added at a time to the filter device, and it is spun at 3750 rpm for 15 minutes. This filtering is repeated until a total final volume of 20 mL of the dextran-coated nanoparticles is obtained.

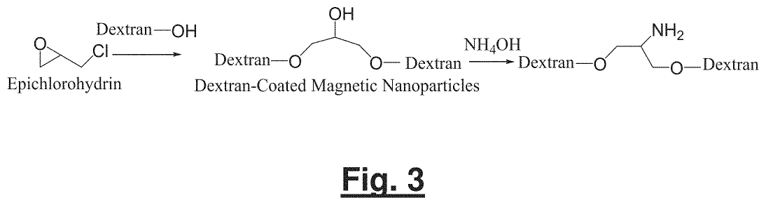

The nanoparticles are then cross-linked with epichlorohydrin. First, 20 mL of a 1 M NaOH solution is added and mixed for 10 minutes. To this, 11 mL of 99% epichlorohydrin is added, and the solution is allowed to mix for 8 hours. This reaction yields nanoparticles at least partially coated with a coating comprising dextran and aminated epichlorohydrin. FIG. 3 shows a reaction scheme for cross-linking the dextran with the epichlorohydrin and for aminating the epichlorohydrin.

Amination of the epichlorohydrin is carried out by adding 45 mL of 30% NH₄OH and stirring a gently capped round bottom flask for about 36 hours. This solution is purified with a membrane against DI water to remove small molecules from the nanoparticles. These particles can be concentrated by magnetic separation and then be placed on a bath sonicator with constant agitation for about 2 to 3 days. Stopping of the sonication and resuspending the aggregates in solution is important for not losing nanoparticles. After sonication is complete, the nanoparticles are completely soluble in aqueous environments and characterization can be performed. The coated nanoparticles are determined to be 20-30 nm in diameter with a sub-5 nm dextran coating. At this point, the coated nanoparticles are also ready to undergo conjugation.

### Fluorophore Conjugation

The coated nanoparticles are conjugated with a Cy7-NHS or Cy5.5-NHS dye molecule (Lumiprobe) to allow for FL/PA/US imaging. Briefly, 100 μL of the dye (10 mM stock) suspended in DMSO is mixed with 400 μL of a sodium citrate buffer (20 mM Na-Citrate, 150 mM NaCl, pH 8.4) to dissolve fully. Next, 1.1 mL of the coated nanoparticles are added to this mixture and placed on a rotor overnight at room temperature. After the conjugation is complete, removal of the excess dye is performed with a PD-10 column against a sodium citrate buffer. The number of dye molecules per nanoparticle is determined to be 11.

### PET/SPECT Chelator Attachment

Similar to the fluorophore conjugation, a DOTA-NHS chelator is next conjugated to the coated nanoparticles to allow for PET imaging. Initially dissolving the chelator in DMSO allows for a similar reaction as previously described. Briefly, 100 μL of the DOTA-NHS (2 mM stock) is first mixed with 300 μL of a sodium citrate buffer (20 mM Na-Citrate, 150 mM NaCl, pH 8.4). To this, 1 mL of the fluorophore-labeled nanoparticles is added slowly, so as to not to induce cloudiness in the solution. The mixture is again placed on a rotor overnight at room temperature and purified with a PD-10 column against a PBS buffer (20 mM Na₂HPO₄, 150 mM NaCl, pH 7.2) when complete. DTPA is conjugated to the core by a similar method using SCN-Bn-CHx-DTPA instead of DOTA-NHS.

### CT Agent Attachment

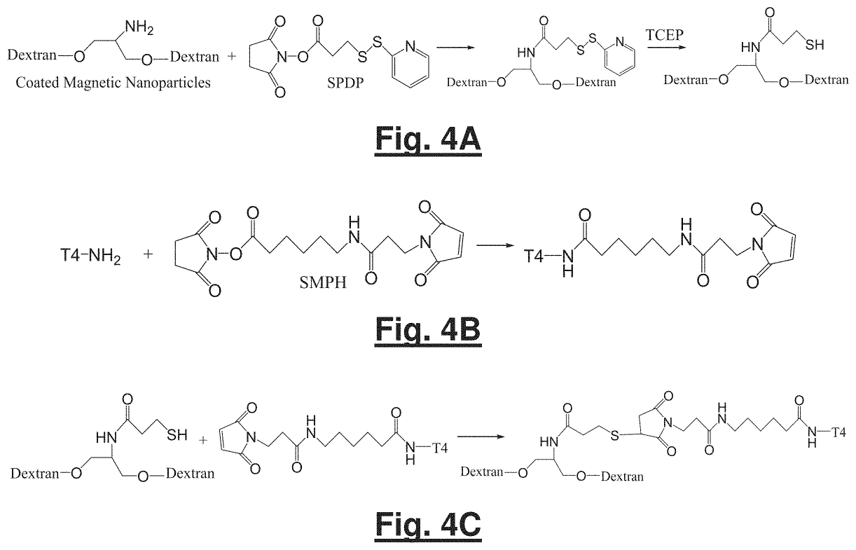

To allow for CT imaging of the nanoparticle constructs, the hormone T4 is conjugated. To obtain this, 100 μL of SPDP (100 mM stock) is first added to 300 μL of the sodium citrate buffer (20 mM Na-Citrate, 150 mM NaCl, pH 8.4). 1 mL of the fluorophore/DOTA-NHS-conjugated coated nanoparticles is then mixed slowly with this to avoid the formation of cloudiness and placed on a rotor at room temperature overnight. These nanoparticles are purified against a PBS buffer (20 mM Na₂HPO₄, 150 mM NaCl, pH 7.2) with a PD-10 column and, once purified, are reacted with 100 μL of TCEP (10 mM stock) to form a reactive thiol group when the T4-linker mixture is prepared. PD-10 column washing against the PBS buffer (20 mM Na₂HPO₄, 150 mM NaCl, pH 7.2) is again performed to remove the reactive small molecules from the magnetic nanoparticles. A reaction scheme for this aspect is shown in FIG. 4A. To prepare the T4-linker mixture, T4 is mixed with an SMPH linker under slightly basic conditions. Briefly, a slurry is made with 153 mg of T4, 200 μL of DMSO, 100 μL of SMPH (10 mM stock), and 800 μL of the sodium citrate buffer. The solution is kept in the dark and placed on a rotor overnight to allow for conjugation. A reaction scheme for this aspect is shown in FIG. 4B. Mixing the TCEP cleaved magnetic nanoparticles with the conjugated slurry at neutral pH gives a final product as shown in the reaction scheme of FIG. 4C. A 50:50 mixture of the nanoparticles in the PBS buffer and the T4 slurry give the best conjugation.

### Results

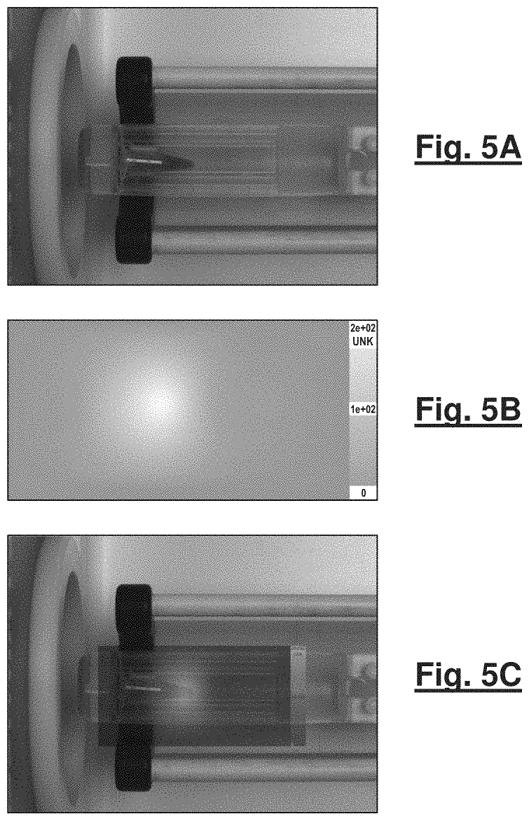

Prior to coupling T4 to the nanoparticles, the nanoparticles are transferred to a microcentrifuge tube and subjected to MPI. FIG. 5A shows a photograph of the microcentrifuge tube containing the nanoparticles, FIG. 5B is an MPI of the microcentrifuge tube containing the nanoparticles, and FIG. 5C is an overlay of the images from FIGS. 5A and 5B.

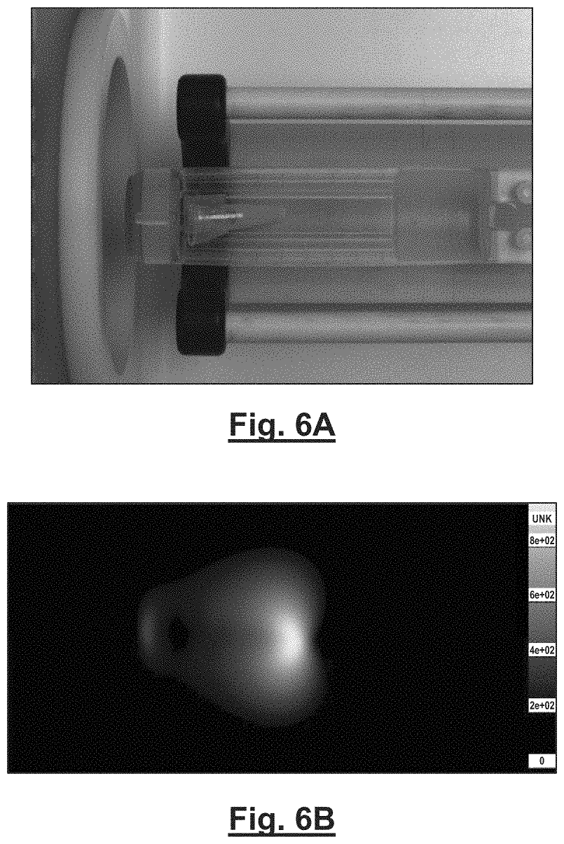

Nanoparticles conjugated with the fluorophore, DOTA-NHS, DTPA, and T4 are transferred to a microcentrifuge tube and subjected to MPI. FIG. 6A shows a photograph of the microcentrifuge tube containing the nanoparticles, and FIG. 6B is an MPI of the microcentrifuge tube containing the nanoparticles.

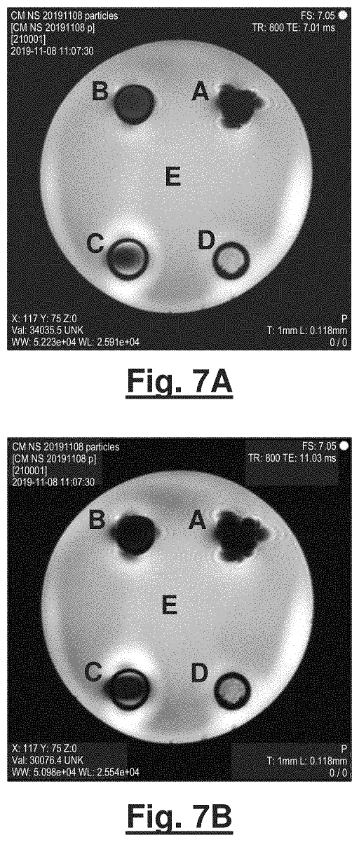

Tubes containing various nanoparticle samples are placed in a vessel containing water and subjected to MRI. The samples include nanoparticles conjugated with fluorophore, DOTA, and NOTA (A), nanoparticles conjugated with fluorophore (B), nanoparticles conjugated with fluorophore and T4 (C), particles prepared by a different protocol (D), and water background (E). FIGS. 7A and 7B are the resulting MRI images, which demonstrate that nanoparticles prepared in accordance with the current technology (A, B, and C) are active under MRI. The nanoparticles prepared by a different protocol (D) showed poor MRI imaging. The water (E) was not active under MRI.

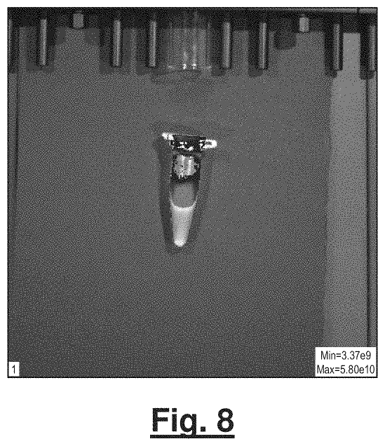

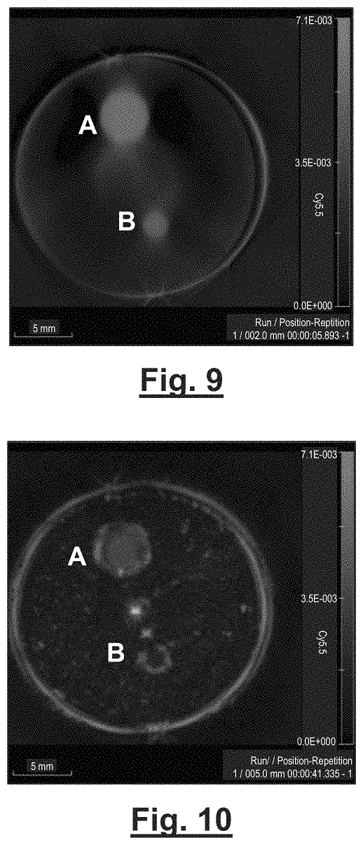

Nanoparticles conjugated with the fluorophore, DOTA-NHS, DTPA, and T4 are transferred to a microcentrifuge tube and subjected to FL imaging in an in vivo imaging system (IVIS). FIG. 8 shows the resulting fluorescence image. A tube containing nanoparticles conjugated with the fluorophore, DOTA-NHS, DTPA, and T4 (A) and a tube containing water (B) are placed in a vessel containing water and subjected to PA imaging and US imaging. FIG. 9 shows an image resulting from the PA imaging, and FIG. 10 shows an image resulting from the US imaging.

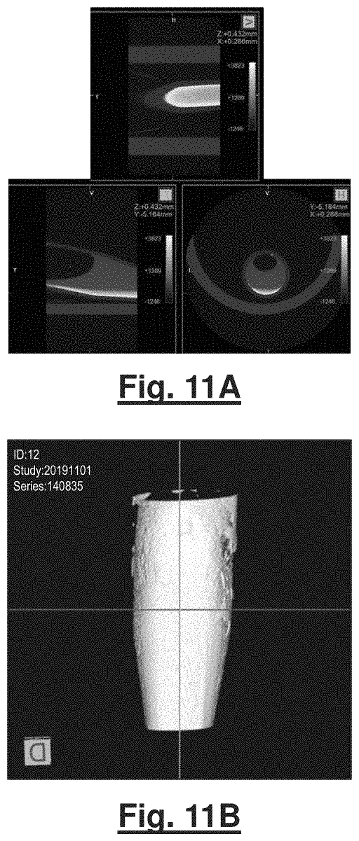

Nanoparticles conjugated with the fluorophore, DOTA-NHS, DTPA, and T4 are transferred to a microcentrifuge tube and subjected to CT. FIG. 11A shows two-dimensional images of slices of the nanoparticles within the microcentrifuge tube from various angles. FIG. 11B shows a three-dimensional image of the nanoparticles contained within the microcentrifuge tube.

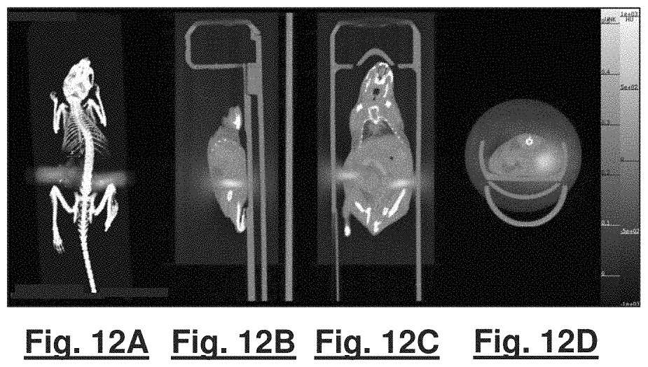

Nanoparticles conjugated with the fluorophore, DOTA-NHS, DTPA, and T4 are intraductally administered to the mammary glands of a mouse. The mouse is then subjected to MPI and CT. FIG. 12A shows an image of the MPI (ductal localization) overlaid with an X-ray image from the CT (Skelton). FIGS. 12B-12D are two-dimensional overlaid images from the MPI CT scans.

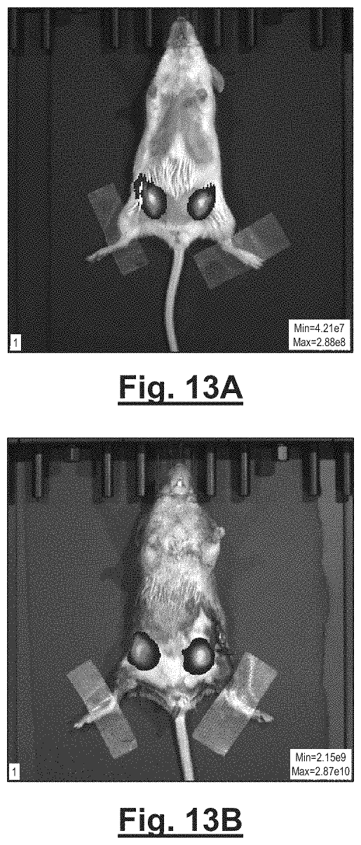

Nanoparticles conjugated with the fluorophore, DOTA-NHS, DTPA, and T4 are intraductally administered to the mammary glands of two mice. The mice are then subjected to FL imaging in an IVIS. Fluorescence images of the two mice are shown in FIGS. 13A and 13B, respectively.

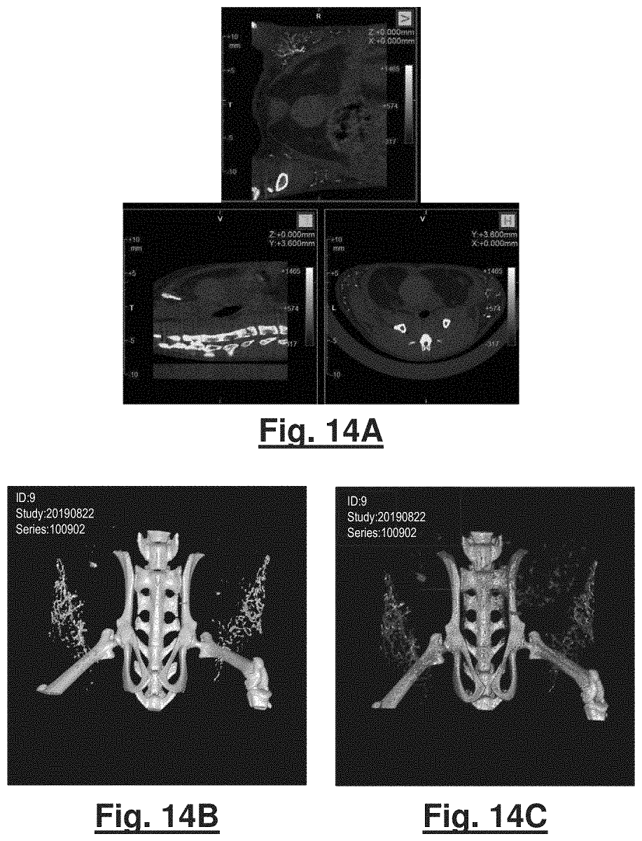

Nanoparticles conjugated with the fluorophore, DOTA-NHS, DTPA, and T4 are intraductally administered to the mammary glands of a mouse. The mouse is then subjected to CT. FIG. 14A includes two-dimensional images of slices of the mouse showing soft tissue of the ductal tree relative to bone tissue. FIGS. 14B and 14C are three-dimensional CT images of the ductal tree relative to a region of the mouse's skeleton.

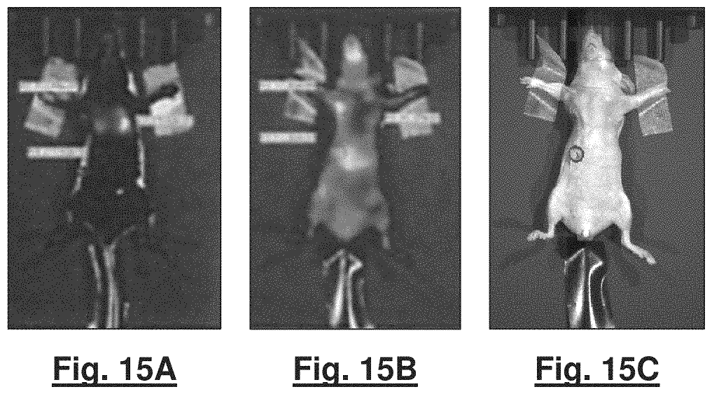

Nanoparticles conjugated with the fluorophore, DOTA-NHS, DTPA, and T4 are intravenously administered to a mouse having a tumor induced by the administration of human breast adenocarcinoma cells (MDA-MB-231). The mouse is then subjected to FL imaging in an IVIS. FIGS. 15A and 15B are fluorescence images taken immediately after the injection and 24 hours after injection, respectively. FIG. 15C is a bioluminescence image of the tumor.

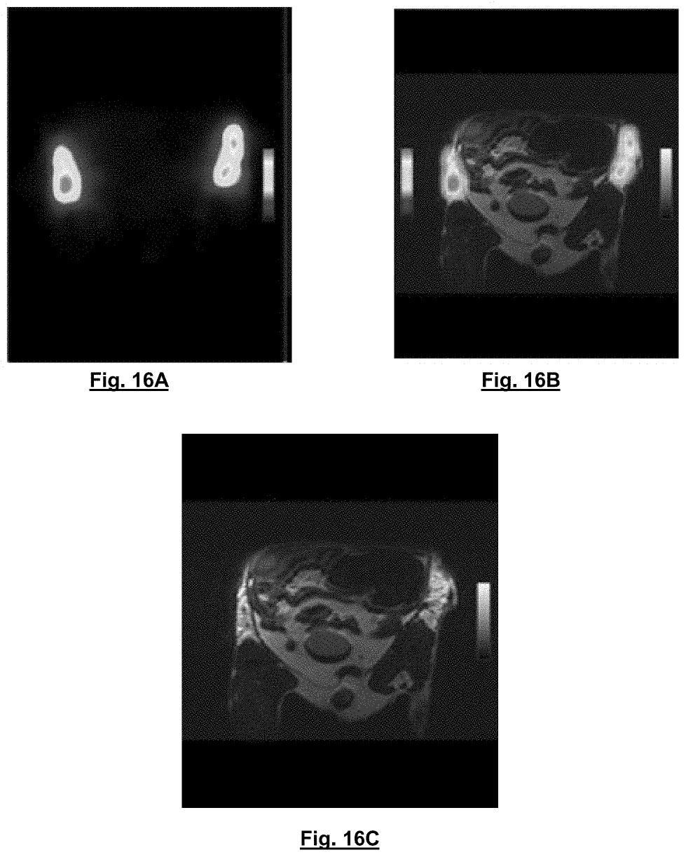

Nanoparticles conjugated with the fluorophore, DOTA-NHS, DTPA and T4 are intraductally administered to the mammary glands of a mouse. The mouse is then subjected to PET/MRI. FIGS. 16A and 16C include two-dimensional images of the slices of the mouse showing soft tissue with MRI and signal generation from the chelated ⁶⁴Cu with PET imaging. FIG. 16B is the two-dimensional overlay of the PET/MR images showing localized signal generation and retention within the ductal tree network.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A nanoparticle comprising:
a magnetic core comprising a magnetic nanocrystal;
a fluorophore coupled to the magnetic core;
at least one chelating compound coupled to the magnetic core, the at least one chelating compound being a compound that chelates copper-64 (⁶⁴Cu), a compound that chelates technetium-99m (^{99m}Tc), or a combination thereof; and
an iodine chelator coupled to the magnetic core.

2. The nanoparticle according to claim 1, wherein the magnetic nanocrystal comprises iron oxide and the magnetic core has a diameter of greater than or equal to about 1 nm to less than or equal to about 50 nm.

3. The nanoparticle according to claim 1, comprising a plurality of magnetic cores, wherein each magnetic core of the plurality comprises a plurality of magnetic nanocrystals.

4. The nanoparticle according to claim 1, wherein the magnetic core is at least partially coated with dextran.

5. The nanoparticle according to claim 4, wherein the fluorophore, the at least one chelating compound, and the iodine chelator are indirectly coupled to the magnetic core by way of the dextran.

6. The nanoparticle according to claim 1, wherein the fluorophore is selected from the group consisting of a cyanine dye, a coumarin, a rhodamine, a xanthene, a quantum dot, derivatives thereof, and combinations thereof.

7. The nanoparticle according to claim 1, comprising the compound that chelates ⁶⁴Cu, the compound that chelates ⁶⁴Cu being S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), or combinations thereof.

8. The nanoparticle according to claim 1, comprising the compound that chelates ^{99m}Tc, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA).

9. The nanoparticle according to claim 1, wherein the iodine chelator is thyroxine (T4), triiodothyronine (T3), or a combination thereof.

10. The nanoparticle according to claim 1, wherein the fluorophore, the compound that chelates ⁶⁴Cu, the compound that chelates ^{99m}Tc, or the combination thereof and the iodine chelator are coupled to the magnetic core by a linker, the linker being aminated epichlorohydrin.

11. The nanoparticle according to claim 8, wherein the iodine chelator is further coupled to the core by succinimidyl 6-((beta-maleimidopropionamido)hexanoate) (SMPH) and succinimidyl 3-(2-pyridyldithio)propionate (SPDP).

12. A nanoparticle comprising:
a single magnetic core having a diameter of greater than or equal to about 20 nm to less than or equal to about 30 nm, the single magnetic core comprising at least one iron oxide nanocrystal;
a coating at least partially covering the single magnetic core, the coating comprising dextran cross-linked with aminated epichlorohydrin; and
an iodine chelator coupled to the aminated epichlorohydrin, the iodine chelator comprising thyroxine (T4), triiodothyronine (T3), or a combination thereof,
wherein the nanoparticle is configured to provide contrast for magnetic resonance imaging (MRI), magnetic particle imaging (MPI), computed tomography (CT), and X-ray imaging.

13. The nanoparticle according to claim 12, further comprising:
a fluorophore coupled to the single magnetic core,
wherein the nanoparticle is further configured to provide contrast for least one of fluorescence (FL) imaging, photoacoustic (PA) imaging, or ultrasound (US) imaging.

14. The nanoparticle according to claim 12, further comprising:
a compound that chelates copper-64 (⁶⁴Cu) coupled to the single magnetic core, the compound that chelates ⁶⁴Cu being selected from the group consisting of S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), and combinations thereof, wherein the nanoparticle is further configured to provide contrast for positron emission tomography (PET).

15. The nanoparticle according to claim 12, further comprising:
a compound that chelates technetium-99m (^{99m}Tc) coupled to the single magnetic core, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA),
wherein the nanoparticle is further configured to provide contrast for single-photon emission computed tomography (SPECT).

16. The nanoparticle according to claim 12, further comprising:
a fluorophore coupled to the single magnetic core;
a compound that chelates copper-64 (⁶⁴Cu) coupled to the single magnetic core, the compound that chelates ⁶⁴Cu being selected from the group consisting of S-2-(4-isothiocyanatobenzyl)-1,4,7,10-tetraazacyclododecane tetraacetic acid (p-SCN-Bn-DOTA), 1,4,7,10-tetraazacyclododecane-1,4,7,10-tetraacetic acid mono-N-hydroxysuccinimide ester (DOTA-NHS-ester), 2-S-(4-isothiocyanatobenzyl)-1,4,7-triazacyclononane-1,4,7-triacetic acid (p-SCN-Bn-NOTA), and combinations thereof; and
a compound that chelates technetium-99m (^{99m}Tc) coupled to the single magnetic core, the compound that chelates ^{99m}Tc being [(R)-2-amino-3-(4-isothiocyanatophenyl)propyl]-trans-(S,S)-cyclohexane-1,2-diamine-pentaacetic acid (SCN-Bn-CHx-DTPA),
wherein the nanoparticle is further configured to provide contrast for positron emission tomography (PET), single-photon emission computed tomography (SPECT), and at least one of fluorescence (FL) imaging, photoacoustic (PA) imaging, or ultrasound (US) imaging.

17. A method of making the nanoparticle according to claim 12, the method comprising:
reacting iron(II) chloride (FeCl₂) with potassium hydroxide (KOH) in water under the flow of an inert gas to form a reaction mixture;
adding hydrogen peroxide (H₂O₂) to the reaction mixture to form a plurality of magnetic cores in the reaction mixture;
applying a magnetic field to the reaction mixture and isolating the plurality of magnetic cores;
contacting the plurality of magnetic cores with dextran to form a dextran coating at least partially covering each magnetic core of the plurality;
cross-linking the dextran with a plurality of epichlorohydrin molecules;
aminating the plurality of epichlorohydrin molecules to form the coating comprising dextran cross-linked with aminated epichlorohydrin on each magnetic core of the plurality; and
conjugating a first portion of the aminated epichlorohydrin molecules on at least a portion of the plurality of magnetic cores with the iodine chelator.

18. The method according to claim 17, further comprising:
conjugating a second portion of the aminated epichlorohydrin molecules with a fluorophore;
conjugating a third portion of the aminated epichlorohydrin molecules with a compound that chelates copper-64 (⁶⁴Cu); and
conjugating a fourth portion of the aminated epichlorohydrin molecules with a compound that chelates technetium-99m (^{99m}Tc).

19. A method of imaging at least one of a tissue or a metabolic process in a subject, the method comprising:
administering to the subject a safe and effective amount of a composition comprising a contrast agent, wherein the contrast agent associates with the at least one of the tissue or the metabolic process, the contrast agent comprising:
a magnetic core comprising a magnetic nanocrystal,
a fluorophore coupled to the magnetic core,
at least one chelating compound coupled to the magnetic core, the at least one chelating compound being a compound that chelates copper-64 (⁶⁴Cu), a compound that chelates technetium-99m (^{99m}Tc), or a combination thereof; and
an iodine chelator coupled to magnetic core;
performing at least one of magnetic resonance imaging (MRI), magnetic particle imaging (MPI), fluorescence (FL) imaging, photoacoustic (PA) imaging, ultrasound (US) imaging, positron emission tomography (PET), single-photon emission computed tomography (SPECT), X-ray imaging, or computed tomography (CT); and
generating an image of the tissue or the metabolic process with contrast provided by the contrast agent.

20. The method according to claim 19, wherein the administering is performed intravenously, intraductally, intrathecally, intramuscularly, intratumorally, or orally.

21. The method according to claim 19, wherein the composition further comprises a pharmaceutically acceptable carrier.

22. The method according to claim 19, comprising:
performing at least two of the MRI, the MPI, the FL imaging, the PA imaging, the US imaging, the PET, the SPECT, the X-ray imaging, or the CT; and
generating at least two images of the tissue or the metabolic process, each of the at least two images comprising contrast provided by the contrast agent.
